(19) United States
(12) Patent Application Publication
(10) Pub. No.: US 2006/0093560 A1
(43) Pub. Date: May 4, 2006

(54) Immediate release film coating

(76) Inventors: Jen-Chi Chen, Morrisville, PA (US); Frank J. Bunick, Randolph, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/977,633

(22) Filed: Oct. 29, 2004

(51) Int. Cl. A61K 9/68; A61K 9/24

(52) U.S. Cl. 424/48; 424/472

(57) ABSTRACT

The present invention is directed to a composition having a shell-forming component that contains a low-molecular weight water-soluble polymer and at least one gum capable of forming or contributing to the formation of thermoreversible gel, wherein the water-soluble polymer has a cloud point in an aqueous system within a temperature range of about 20° C. and about 90° C. The compositions are particularly suited for forming shell coatings on medicinal dosage forms.

### FIELD OF THE INVENTION

The present invention relates to the field of film-forming or gel-forming compositions, particularly towards substitutes for gelatin, and particularly for non-gelatin film coatings for oral delivery of medications or diet supplements.

### BACKGROUND OF THE INVENTION

Carrageenan is a natural hydrocolloid, a polysaccharide hydrocolloid, which is derived from seaweed. It comprises a carbohydrate polymer of repeating sugar units, which is linear, without significant numbers of branches or substitutions. Most, if not all, of the galactose units on a Carrageenan molecule possess a sulfate ester group. The exact position of the sulfate groups, the cations on the sulfate groups, and the possible presence of an anhydrous bridge on the molecule differentiates the various types of Carrageenan. There are three distinct types of Carrageenan: kappa, iota and lambda forms of Carrageenan. These various forms can significantly vary in properties, as exemplified by the fact that lambda Carrageenan in solution is unable to associate into a structure, so that it cannot gel, but may act as a thickener. Both kappa and iota Carrageenan are able to gel. Kappa Carrageenan is known to form gels in the presence of potassium cations. These gels tend to be brittle and exhibit syneresis (contraction and release of entrapped liquid) as the gel shrinks. Iota Carrageenan tends to react strongly to calcium cations and forms a more tender, flexible gel than kappa Carrageenan that is not as susceptible to syneresis.

It is known to coat tablets with hydrocolloids selected from the group consisting of locus beam gum, guar gum, carrageenan and mixtures thereof as shown in published PCT application WO 01/26633. The application does not indicate what form of carrageenan gum was used or incorporate other film-forming or gelling agents. U.S. Pat. No. 6,214,376 discloses a film composition for capsules comprising a water-dispersible or water soluble plasticizer and carrageenan, with the carrageenan comprising at least 50% by weight of all gums of a k-carrageenan and wherein carrageenan comprising at least 50% by weight of all gums that form or contribute to the formation of the thermoreversible gels. The compositions described therein do not contain a cellulosic polymer.

A variety of cellulosic polymers are known to be useful in the preparation of dosage forms. They are often combined with other polymers and thickeners and used as coatings or shells for dosage forms. For example, WO 01/32150 discloses an edible, hardenable coating composition containing microcrystalline cellulose, carrageenan, and at least one of a strengthening polymer, a plasticizer, a surface-active agent or a combination thereof. Similarly, published PCT application WO 00/45794 discloses an edible, hardenable coating composition containing microcrystalline cellulose, carrageenan and either a strengthening polymer, a plasticizer or both.

Published U.S. patent application 2004/0129174 describes compositions comprising a high molecular weight, water soluble polymer having a cloud point from about 20 to about 90 C and at least one carrageenan. The compositions can be used as a component of a dosage form, such as a shell, to provide burst release of the active ingredient contained therein.

U.S. Pat. No. 3,962,482 describes clear, elastic, water gels and gel-forming compositions that are based on potassium-sensitive carrageenan in the form of an alkali metal or an ammonium salt and a water-soluble potassium salt. Addition to the composition of calcium-sensitive carrageenan, also in the form of an alkali metal or an ammonium salt, imparts freedom from syneresis. The water gels and the gel-forming compositions are characterized by essentially complete freedom from polyvalent metal cations.

U.S. Pat. No. 5,089,307 discloses heat-sealable, edible films comprising at least a film layer containing a water-soluble polysaccharide as the principal component, or comprising at least (a) a film layer as described above and (b) a subfilm layer containing an alkali metal salt of casein, soybean protein or a combination of soybean protein and gelatin, as the principal component.

U.S. Pat. No. 5,002,934 describes aqueous gels, gel-forming compositions and composites containing the same, comprising carrageenan and a cation of such a type and in such a concentration that the gel has a transition midpoint temperature below 45 C and a yield stress of at least 0.5 kN/m² at 5 C.

U.S. Pat. No. 4,276,320 describes a method and a kappa carrageenan composition for making a water dessert gel having a controlled melting temperature so as to soften or melt within the mouth of the consumer and providing for excellent flavor release, good mouth feel and containing only kappa carrageenan, and sodium salt of a sequestering agent with ionizable potassium in amounts sufficient to sequester all polyvalent cations present.

U.S. Pat. No. 3,956,173 describes cold-water gellable compositions that are prepared based on the sodium salt of kappa-carrageenan and a potassium salt. Gelation is controlled so that good quality gels result by encapsulating the potassium salt in a water-soluble hydroxypropyl cellulose.

WO 00/40223 relates to a composition comprising hydroxypropylcellulose and at least one anionic polymer such as carboxymethyl ether salts of cellulose, methacrylic acid polymers and copolymers, carboxyvinyl polymers and copolymers, alginic acid salts, pectinic acid salts, pectic acid salts, carrageenan, agar and carboxylic acid salts of polysaccharides. The ratio of hydroxypropylcellulose to anionic polymer is from 1:20 to 20:1. The composition is used as an aqueous solution to coat substrates.

U.S. Pat. No. 6,358,525 B1 discloses a pharmaceutical composition containing a medicament and a blend of two components. The first component is hydroxypropylcellulose and the second component is at least one other polymer selected from a group that includes carrageenan, agar, and gellan gum. The pharmaceutical composition is formed into a tablet that may be coated with a conventional coating material.

U.S. Pat. No. 6,245,356 B1 relates to a sustained release, oral, solid dosage form comprising agglomerated particles of a therapeutically active medicament in amorphous form, a gelling agent, an ionizable gel strength enhancing agent and an inert diluent. The gelling agent preferably comprises xanthan gum and locust bean gum, but may alternatively comprise alginates, carrageenan, pectin, and other compounds. The ionizable gel strength-enhancing agent may be a monovalent or multivalent metal cation. The active medicament in amorphous form, gelling agent, ionizable gel strength enhancing agent and an inert diluent are mixed or granulated together and formed into a tablet.

Applicants have now discovered that a composition comprising a combination of a low molecular weight, water soluble polymer and at least one gum capable of forming or contributing to forming a thermoreversible gel may be used as a component of a dosage form, for example as the shell of a dosage form containing active ingredient in an underlying core. The low molecular weight, water soluble polymer and at least one gum can be dispersed in water, along with other ingredients, at a temperature above the cloud point of the low molecular weight, water soluble polymer, leaving the low molecular weight, water soluble polymer undissolved and the viscosity of the dispersion manageable. The dispersion flows easily, and sets quickly and strongly at a relatively high temperature due to the presence of the at least one gum capable of forming or contributing to the forming of a thermoreversible gel.

### SUMMARY OF THE INVENTION

The present invention is directed to a composition having a shell-forming component that contains a low-molecular weight water-soluble polymer and at least one gum capable of forming or contributing to the formation of thermoreversible gel. The water-soluble polymer has a cloud point in an aqueous system within a temperature range of about 20° C. and about 90° C. The gum can be a blend of gums capable of forming or contributing to the formation of thermoreversible gel that is at least 50% by weight of a Kappa-carrageenan.

In an alternative embodiment, the shell-forming component comprises 20 to 75 weight percent of the low molecular weight, water-soluble polymer as a percentage of the dried film and 25 to 80 weight percent of the at least one gum as a percentage of the dried film. The low molecular weight, water soluble polymer can be selected from the group consisting of hydroxypropylmethyl cellulose, hydroxypropyl cellulose, methyl cellulose and mixtures thereof. Alternatively, the low molecular weight, water soluble polymer can contain hydroxypropyl methylcellulose having a viscosity from about 3 to about 80 mPa s in 2% aqueous solution at 25° C. In a further alternative, the low molecular weight, water soluble polymer can contain hydroxypropyl methylcellulose having a viscosity from about 3 to about 50 mPa s in 2% aqueous solution at 25° C. Still further, the low molecular weight, water soluble polymer can contain at least 75% by weight of the total weight of water soluble polymer in the composition as hydroxypropyl methylcellulose having a viscosity from about 3 to about 50 mPa s in a 2% aqueous solution at 25° C. The shell-forming component can optionally further include a gelling salt.

The composition can be characterized by having a water-soluble polymer that has a cloud point in an aqueous system within a temperature range of about 30° C. and about 80° C. Alternatively, the water-soluble polymer can have a cloud point in an aqueous system within a temperature range of about 35° C. and about 70° C.

The present invention also relates to a composition wherein a percentage of active ingredient dissolved from the finished dosage form after application and drying of the shell is not less than 90% of a percentage of active ingredient dissolved at any time point of the dissolution rate of an equivalent uncoated core, according to a preferred method of analysis for said active. Such analysis should be conducted within a reasonable time not to exceed 24 hours from completion of the drying step.

The present invention also relates to a composition wherein a percentage of active ingredient dissolved from the finished dosage form upon storage conditions of 40° C. and 75% relative humidity for up to 6 months is not less than 90% of the dissolved active at any time point of the dissolution rate of an equivalent uncoated core, according to a preferred method of analysis for said active.

The present invention further relates to composition wherein the degradation of the active ingredient is not more than 1% as measured by the chemically degraded derivative compound of the active ingredient upon application and drying of the shell. Such analysis should be conducted within a reasonable time not to exceed 24 hours from completion of the drying step.

The present invention further relates to a composition wherein the degradation of the active ingredient is not more than 1% as measured by the chemically degraded derivative compounds of the active ingredient at storage conditions of 40° C. and 75% relative humidity for up to 6 months.

The present invention further relates to a dosage form comprising a shell that is formed from a low-molecular weight water-soluble polymer and at least one gum capable of forming or contributing to the formation of thermoreversible gel. The water-soluble polymer has a cloud point in an aqueous system within a temperature range for the aqueous system of about 20° C. and about 90° C. The water-soluble polymer has a cloud point in an aqueous system within a temperature range of about 30° C. and about 80° C. Alternatively, the water-soluble polymer can have a cloud point in an aqueous system within a temperature range of about 35° C. and about 70° C. The gum can be a blend of gums capable of forming or contributing to the formation of thermoreversible gel that is at least 50% by weight of a Kappa-carrageenan.

In an alternative embodiment, the shell-forming component of said dosage form comprises 20 to 75 weight percent of the low molecular weight, water-soluble polymer as a percentage of the dried film and 25 to 80 weight percent of the at least one gum as a percentage of the dried film. The low molecular weight, water soluble polymer for said shell component can be selected from the group consisting of hydroxypropylmethyl cellulose, hydroxypropyl cellulose, methyl cellulose and mixtures thereof. The low molecular weight, water soluble polymer can contain hydroxypropyl methylcellulose having a viscosity from about 3 to about 80 mPa s in 2% aqueous solution. Alternatively, the low molecular weight, water soluble polymer can contain hydroxypropyl methylcellulose having a viscosity from about 3 to about 50 mPa s in 2% aqueous solution. Still further, the low molecular weight, water soluble polymer can contain at least 75% by weight of the total weight of water soluble polymer in the composition as hydroxypropyl methylcellulose having a viscosity from about 3 to about 50 mPa s in 2% aqueous solution.

The present invention also relates to a process for preparing a dosage form by coating a core containing a pharmaceutical active ingredient with any of the compositions described above.

The present invention also relates to a process for preparing a core and shell dosage form by forming a compressed core containing at least one pharmaceutical active ingredient in compression tableting machine and coating the compressed core with any of the compositions described above.

The present invention also relates to a process for preparing a core and shell dosage form by forming a solid, compressed core containing at least one pharmaceutical active ingredient in a tableting machine, introducing the compressed core into a mold cavity and injecting any of the composition described above into the mold cavity to coat at least a portion of the compressed core.

The present invention also relates to a process for preparing a core and shell dosage form by forming a solid, compressed core containing at least one pharmaceutical active ingredient in a tableting machine, introducing the compressed core into a mold cavity, injecting any of the compositions described above into the mold cavity to coat at least a portion of the compressed core, rotating the mold cavity, and injecting a liquid curable composition into said mold to coat at least a second portion of the compressed core.

The present invention also relates to a dosage form comprising a core, having a shell at least on a portion thereof, wherein the shell has a thickness from about 10 to about 80 microns prepared by introducing said core into a mold cavity and injecting any of the compositions described above into the mold cavity to coat at least a portion of the core. The foregoing dosage form can be ejected from the mold cavity following injection of the composition into the mold cavity in such a manner that the injection of said composition and ejection of the dosage form takes 6 seconds or less.

The present invention also relates a composition that consists essentially of: a) 20 to 75 weight percent of hydroxypropyl methylcellulose having a viscosity from about 3 to about 80 mPa s in 2% aqueous solution; b) 25 to 80 weight percent of a gum component comprising at least 50% by weight of Kappa-carrageenan. Alternatively, the present invention relates to an aqueous dispersion comprising: a) 1 to 11 weight percent of a low molecular weight, water soluble polymer that has a cloud point in an aqueous systems within a temperature range for the aqueous system of about 20° C. and about 80° C.; b) 1.3 to 12 weight percent of a gum component comprising at least 50% by weight of Kappa-carrageenan; and d) about 85-95 weight percent water.

### DETAILED DESCRIPTION OF THE INVENTION

As used herein, the term “dosage form” applies to any solid object or semi-solid object designed to contain a specific pre-determined amount (dose) of a certain ingredient, for example an active ingredient as defined below. Suitable dosage forms may be pharmaceutical drug delivery systems, including those for oral administration, buccal administration, rectal administration, topical or mucosal delivery, or subcutaneous implants, or other implanted drug delivery systems; or compositions for delivering minerals, vitamins and other nutraceuticals, oral care agents, flavorants, and the like. Preferably the dosage forms of the present invention are considered to be solid, however they may contain liquid or semi-solid components. In a particularly preferred embodiment, the dosage form is an orally administered system for delivering a pharmaceutical active ingredient to the gastro-intestinal tract of a human.

Suitable active ingredients for use in this invention include for example pharmaceuticals, minerals, vitamins and other nutraceuticals, oral care agents, flavorants and mixtures thereof. Suitable pharmaceuticals include analgesics, anti-inflammatory agents, antiarthritics, anesthetics, antihistamines, antitussives, antibiotics, anti-infective agents, antivirals, anticoagulants, antidepressants, antidiabetic agents, antiemetics, antiflatulents, antifungals, antispasmodics, appetite suppressants, bronchodilators, cardiovascular agents, central nervous system agents, central nervous system stimulants, decongestants, oral contraceptives, diuretics, expectorants, gastrointestinal agents, migraine preparations, motion sickness products, mucolytics, muscle relaxants, osteoporosis preparations, polydimethylsiloxanes, respiratory agents, sleep-aids, urinary tract agents and mixtures thereof.

Suitable oral care agents include breath fresheners, tooth whiteners, antimicrobial agents, tooth mineralizers, tooth decay inhibitors, topical anesthetics, mucoprotectants, and the like.

Suitable flavorants include menthol, peppermint, mint flavors, fruit flavors, chocolate, vanilla, bubblegum flavors, coffee flavors, liqueur flavors and combinations and the like.

In another embodiment, the active ingredient is selected from analgesics, anti-inflammatories, and antipyretics, e.g. non-steroidal anti-inflammatory drugs (NSAIDs), including propionic acid derivatives, e.g. ibuprofen, naproxen, ketoprofen and the like; acetic acid derivatives, e.g. indomethacin, diclofenac, sulindac, tolmetin, and the like; fenamic acid derivatives, e.g. mefanamic acid, meclofenamic acid, flufenamic acid, and the like; biphenylcarbodylic acid derivatives, e.g. diflunisal, flufenisal, and the like; and oxicams, e.g. piroxicam, sudoxicam, isoxicam, meloxicam, and the like. In one particular embodiment, the active ingredient is selected from propionic acid derivative NSAID, e.g. ibuprofen, naproxen, flurbiprofen, fenbufen, fenoprofen, indoprofen, ketoprofen, fluprofen, pirprofen, carprofen, oxaprozin, pranoprofen, suprofen, and pharmaceutically acceptable salts, derivatives, and combinations thereof In another particular embodiment of the invention, the active ingredient may be selected from acetaminophen, acetyl salicylic acid, ibuprofen, naproxen, ketoprofen, flurbiprofen, diclofenac, cyclobenzaprine, meloxicam, rofecoxib, celecoxib, and pharmaceutically acceptable salts, esters, isomers, and mixtures thereof.

In another embodiment of the invention, the active ingredient may be selected from pseudoephedrine, phenylpropanolamine, chlorpheniramine, dextromethorphan, diphenhydramine, guaifenesin, astemizole, terfenadine, fexofenadine, loratadine, desloratadine, cetirizine, mixtures thereof and pharmaceutically acceptable salts, esters, isomers, and mixtures thereof.

The active ingredient or ingredients are present in the dosage form in a therapeutically effective amount, which is an amount that produces the desired therapeutic response upon oral administration and can be readily determined by one skilled in the art. In determining such amounts, the particular active ingredient being administered, the bioavailability characteristics of the active ingredient, the dosing regimen, the age and weight of the patient, and other factors must be considered, as known in the art. Typically, the dosage form comprises at least about 1 weight percent, preferably, the dosage form comprises at least about 5 weight percent, e.g. at least about 25 weight percent of a combination of one or more active ingredients. In one embodiment, a core comprises a total of at least about 50 weight percent, e.g. at least about 70 weight percent, say at least about 80 weight percent (based on the weight of the core) of one or more active ingredients.

The active ingredient or ingredients may be present in the dosage form in any form. For example, the active ingredient may be dispersed at the molecular level, e.g. melted or dissolved, within the dosage form, or may be in the form of particles, which in turn may be coated or uncoated. If the active ingredient is in form of particles, the particles (whether coated or uncoated) typically have an average particle size of about 1-2000 microns. In one embodiment, such particles are crystals having an average particle size of about 1-300 microns. In another embodiment, the particles are granules or pellets having an average particle size of about 50-2000 microns, for example about 50-1000 microns, say about 100-800 microns.

The composition of the present invention comprises a shell-forming component in an aqueous carrier system for a dosage form having immediate release properties. Dissolution testing for immediate release dosage form is usually conducted on equipment that conforms USP requirements and by a validated analysis method. The dissolution time is generally 30 to 60 minutes, with a single time point for pharmacopeial purposes. Typical specifications for the amount of active ingredient dissolved, expressed as a percentage of the labeled content (Q), are in the range of 70% to 80% Q dissolved. The shell-forming component comprises a combination of a low molecular weight, water-soluble polymer and at least one gelling gum. The gelling gum can comprise one or more carrageenan gums, and optionally gellan gum and/or a lubricant such as glyceryl monostearate.

One embodiment of the present invention includes a core in the form of a compressed tablet, a capsule shell, or a molded tablet having a shell that is substantially free of pores having a diameter of 0.5 to 5.0 microns.

The resulting dosage form is preferably a compressed core having a shell that is preferably substantially free of pores having a diameter of 0.5 to 5.0 microns. Alternatively, the shell-forming composition can be used as a component of a pharmaceutical dosage form, a portion of a shell of a dosage form, the core of a dosage form, or a portion of the core of a dosage form. The use of a low molecular weight, water soluble cellulosic polymer as a part of the shell is important so that the resulting dosage form retains immediate release properties for at least a portion of the underlying compressed core, while, in a preferred embodiment, protecting water-sensitive ingredients in the core from the moisture retained by in the shell coating and/or found in the surrounding environment.

Typically, when water penetrates the core of a dosage form, the dissolution rate of the active ingredient can be adversely affected and decreased. The shell composition in the present invention prevents the dissolution rate from decreasing, wherein the portion of the active ingredient dissolved from the finished dosage form at any timepoint according to a preferred method for said active is not less than 90% of the dissolution rate of the uncoated core. Stability of dissolution rate in the dosage form is present immediately upon manufacture and at accelerated storage conditions up to 6 months at 40° C. and 75% relative humidity. Dissolution rate is defined as the percent of active ingredient released over time, wherein the active dissolves in a media specified by, and: is analyzed by a method at specified timepoints defined by, the United States Pharmacopoeia for said active.

Typically, when water penetrates the core of a dosage form, the chemical stability of the active ingredient can be adversely affected. The shell composition in the present invention prevents the chemical stability from being affected by preventing water from penetrating the core. Poor chemical stability is defined as the degradation of the active ingredient up to 1% as measured by the chemically degraded derivative compounds of the active ingredient. Chemical stability of the active ingredient through the prevention of degradation in the dosage form is present immediately upon manufacture and at accelerated storage conditions up to 6 months of 40° C. and 75% relative humidity.

Shell-forming compositions used in injection molding systems typically contain a relatively high percentage of water and are applied directly over the cores. The ability of a composition to protect water sensitive ingredients is a significant advantage for when making dosage forms having coatings or shells that have been injected molded over compressed cores or otherwise contain a large amount of water. Hence, these compositions create greater challenges in preventing hydrolysis reactions.

The shell-forming compositions described herein are also preferably applied onto or injected into the molds at relatively high temperatures that are above the cloud point of the dispersed low molecular weight polymers. The preferred low molecular weight polymers exhibit a desired thermal dissolution profile such that as the temperature (within conventional -operating conditions of about 20° C. to 100° C.) of the carrier system falls, the low molecular weight polymers begin the dissolution process and form an interconnecting network of polymer branches. In other words, there is greater dissolution at lower temperature than at high temperatures. The dissolution process for the dispersed polymer draws water into their network and away from the coated core. Since coatings are applied or injected at elevated temperatures and then allowed to cool, the low molecular weight polymer must exhibit the desired thermal dissolution profile to draw water away from the core at the appropriate time in the coating step.

Examples of water sensitive ingredients commonly found in pharmaceutical tablets include active ingredients, such as, disintegrants, such as sodium starch glycolate, cross-linked polyvinylpyrrolidone, cross-linked carboxymethylcellulose, starches, microcrystalline cellulose, and the like, binders, such as starch, polyvinylpyrrolidone, hydroxypropylcellulose, and hydroxypropylmethylcellulose, excipients, such as water-soluble compressible carbohydrates such as sugars, which include dextrose, sucrose, maltose, and lactose, sugar-alcohols, which include mannitol, sorbitol, maltitol, xylitol, starch hydrolysates, which include dextrins, and maltodextrins, and the like, water insoluble plastically deforming materials such as microcrystalline cellulose or other cellulosic derivatives.

Examples of suitable low molecular weight, water-soluble polymers that exhibit the desired dissolution temperature profile include hydroxypropylmethyl cellulose, hydroxypropyl cellulose, methylcellulose and mixtures thereof.

In one embodiment, the low molecular weight, water-soluble polymer comprises hydroxypropyl methylcellulose having a viscosity from about 3 to about 80 mPa s in 2% aqueous solution. In a further embodiment, the low molecular weight, water soluble polymer comprises hydroxy propyl methylcellulose having a viscosity of about 3 to 50 mPa s in 2% aqueous solution. In a further embodiment, the low molecular weight, water soluble polymer comprises hydroxy propyl methylcellulose having a viscosity of about 3 to 20 mPa s in 2% aqueous solution. In a further embodiment, the low molecular weight, water soluble polymer comprises hydroxy propyl methylcellulose having a viscosity of about 3 to 12 mPa s in 2% aqueous solution.

In one embodiment, the low molecular weight, water soluble polymer comprises hydroxypropyl cellulose having a viscosity from about 3 to about 80 mPa s in 2% aqueous solution. In a further embodiment, the low molecular weight, water soluble polymer comprises hydroxypropyl cellulose having a viscosity of about 3 to 50 mPa s in 2% aqueous solution. In a further embodiment, the low molecular weight, water soluble polymer comprises hydroxypropyl cellulose having a viscosity of about 3 to 20 mPa s in 2% aqueous solution. In a further embodiment, the low molecular weight, water soluble polymer comprises hydroxypropyl cellulose having a viscosity of about 3 to 12 mPa s in 2% aqueous solution.

In one embodiment, the low molecular weight, water soluble polymer comprises methylcellulose having a viscosity from about 3 to about 80 mPa s in 2% aqueous solution. In a further embodiment, the low molecular weight, water soluble polymer comprises methylcellulose having a viscosity of about 3 to 50 mPa s in 2% aqueous solution. In a further embodiment, the low molecular weight, water soluble polymer comprises methylcellulose having a viscosity of about 3 to 20 mPa s in 2% aqueous solution. In a further embodiment, the low molecular weight, water soluble polymer comprises methylcellulose having a viscosity of about 3 to 12 mPa s in 2% aqueous solution.

The shell-forming component of the composition also comprises at least gum that forms or contributes to the formation of thermoreversible gels. It is desirable to be able to distinguish amongst the various types of gums preferred and tolerated in the practice of the present invention. Gums (hydrocolloids) that form thermoreversible gels or contribute to the formation of thermoreversible gels include, for example, Kappa-carrageenan, iota-carrageenan, xanthan gum, gellan gum, and mannan gums (such as locust bean gum, konjac gum, tara gum and cassia gum). The specific words used in the description of “or contribute to the formation of thermoreversible gels” are important because some of these gums, such as the mannan gums and xanthan gum, do not form thermoreversible gels by themselves, but they form thermoreversible gels with carrageenan through a synergistic effect. Gums (hydrocolloids) that do not form thermoreversible gels include dextrins (including maltodextrin), proteins, gum arabic and polyvinylpyrrolidone (e.g., Povidone). The latter gums may simply be film formers (such as gum arabic and Povidone) or both film formers and formers of non-thermoreversible (heat stable) gels (such as various plant proteins, for example, soy protein). The term ‘thermoreversible gum’ therefore refers to a gum the gel of which is thermoreversible or contributes to the formation of thermoreversible gels with Kappa-carrageenan.

Optionally, mannan gums (e.g., locust bean gum, konjac gum, and tara gum) which have a synergistic gelling effect with Kappa-carrageenan can be added to increase gel strength and elasticity. Also, part of Kappa-carrageenan may be substituted by iota-carrageenan (up to a maximum of 50% or 25% by weight of the Kappa-carrageenan) to form “softer” and more elastic gels. Mechanical properties of carrageenan films can also be improved through a synergistic effect with added mixtures of xanthan gum (a microbial gum) and locust bean gum.

Accordingly, the composition in one embodiment comprises about 20 to about 75 weight percent of a low molecular weight, water soluble polymer having the desired thermal dissolution profile, 25 to 80 weight percent of at least one gum capable of forming or contributing to the formation of a thermoreversible gel.

In a further embodiment, the composition in one embodiment comprises about 20 to about 75 weight percent of a low molecular weight, water soluble polymer having the desired thermal dissolution profile, 25 to 80 weight percent of at least one gum capable of forming or contributing to the formation of a thermoreversible gel wherein at least 50% of gums in the overall composition are Kappa-carrageenan.

In a further embodiment, the composition in one embodiment comprises about 20 to about 75 weight percent of a low molecular weight, water soluble polymer having the desired thermal dissolution profile, 25 to 80 weight percent of at least one gum capable of forming or contributing to the formation of a thermoreversible gel wherein at least 75% of gums in the overall composition are Kappa-carrageenan.

In a further embodiment, the composition in one embodiment comprises about 20 to about 75 weight percent of a low molecular weight, water soluble polymer having the desired thermal dissolution profile, 25 to 80 weight percent of at least one gum capable of forming or contributing to the formation of a thermoreversible gel wherein at least 90% of gums in the overall composition are Kappa-carrageenan.

In one embodiment, the composition further comprises gellan gum, preferably in the range of about 0.5 to about 5 weight percent of the composition. Examples of useful gellan gums include unclarified low acyl, clarified low acyl, and unclarified high acyl gellan gum and combinations thereof. In one embodiment, the gellan gum comprises unclarified high acyl gellan gum

Accordingly, the composition in one embodiment comprises about 20 to about 75 weight percent of a low molecular weight, water soluble polymer having the desired thermal dissolution profile, 25 to 80 weight percent of a blend of gums capable of forming or contributing to the formation of a thermoreversible gel wherein at least 50% of the gums in the overall composition are Kappa-carrageenan and 0.5 to 5 weight percent gellan gum.

In a further embodiment, the composition in one embodiment comprises about 20 to about 75 weight percent of a low molecular weight, water soluble polymer having the desired thermal dissolution profile, 25 to 80 weight percent of a blend of gums capable of forming or contributing to the formation of a thermoreversible gel wherein at least 75% of the gums in the overall composition are Kappa-carrageenan and 0.5 to 5 weight percent gellan gum.

In a further embodiment, the composition in one embodiment comprises about 20 to about 75 weight percent of a low molecular weight, water soluble polymer having the desired thermal dissolution profile, 25 to 80 weight percent of a blend of gums capable of forming or contributing to the formation of a thermoreversible gel wherein at least 90% of the gums in the overall composition are Kappa-carrageenan and 0.5 to 5 weight percent gellan gum.

In another embodiment, the composition consists essentially of a) 20 to 75 weight percent of hydroxypropyl methylcellulose having a viscosity from about 3 to about 50 mPa s in 2% aqueous solution; b) 25 to 80 weight percent of at least one gum capable of forming or contributing to the formation of a thermoreversible gel and c) up to 10 weight percent of an ionic gelling salt, such as potassium chloride. In another embodiment, the composition further comprises a lubricant, preferably in the range of about 0.5 to about 30 weight percent of the composition.

In another embodiment, the composition consists essentially of a) 20 to 75 weight percent of hydroxypropyl cellulose having a viscosity from about 3 to about 50 mPa s in 2% aqueous solution; b) 25 to 80 weight percent of at least one gum capable of forming or contributing to the formation of a thermoreversible gel and c) up to 10 weight percent of an ionic gelling salt, such as potassium chloride. In another embodiment, the composition further comprises a lubricant, preferably in the range of about 0.5 to about 30 weight percent of the composition.

In another embodiment, the composition consists essentially of a) 20 to 75 weight percent of methylcellulose having a viscosity from about 3 to about 50 mPa s in 2% aqueous solution; b) 25 to 80 weight percent of at least one gum capable of forming or contributing to the formation of a thermoreversible gel and c) up to 10 weight percent of an ionic gelling salt, such as potassium chloride. In another embodiment, the composition further comprises a lubricant, preferably in the range of about 0.5 to about 30 weight percent of the composition.

The lubricant may be, for example, glyceryl monostearate, glyceryl palmitostearate, glycerol monooleate, hydrogenated vegetable oil, type I, magnesium stearate, and talc. Preferably, the lubricant is glyceryl monostearate.

In another embodiment, the shell-forming component further comprises active ingredient. When active ingredient is present, the level of low molecular weight water soluble polymer in the shell-forming component is adjusted downward by the amount of the active ingredient. In one particular embodiment, the shell-forming component comprises up to about 80 weight percent of at least one active ingredient; about 15 to about 95 weight percent of a low molecular weight, water soluble polymer having the desired thermal dissolution profile; and about 5 to about 85 weight percent of at least one gum capable of forming or contributing to the formation of a thermoreversible gel.

The composition, whether used as a shell, portion of a shell, i.e. “shell portion,” core, core portion, or as a dosage form per se, may comprise other optional ingredients. In one embodiment, the composition also comprises an inorganic cation as an ionic gelling aid. Suitable inorganic cations include pharmaceutically acceptable monovalent, divalent, and trivalent cations. For example, the inorganic cation may be selected from the group consisting of potassium cations, calcium cations, and mixtures thereof. For the Kappa carrageenan, potassium chloride is preferred should additional film strength be desired.

In another embodiment, the composition also comprises a water-insoluble polymer. Suitable water-insoluble polymers include of ethyl cellulose, cellulose acetate, cellulose acetate butyrate and mixtures thereof.

In one embodiment, a dosage form according to the invention comprises a core at least partially surrounded by a shell or a shell portion formed from the compositions described above. Such shell may comprise about I to about 75, or about 2 to about 24, or about 5 to about 15, weight percent of the total weight of the dosage form. The average thickness of the shell or shell portion may be in the range of about 50 to about 500 microns.

The shell may completely surround the core, or only partially surround the core. Moreover, only one shell portion may comprise the composition of the invention, as further discussed below. For example, in one embodiment a shell comprising a first shell portion and a second shell portion surrounds the core, and the first shell portion comprises the composition of the present invention, while the second shell portion is compositionally different from the first shell portion. In embodiments wherein a first shell portion of a dosage form comprises the composition of the present invention, the weight of said first shell portion may be from about 1 to about 75, e.g. about 1 to about 25, or about 1 to about 10 percent of the weight of the dosage form.

In embodiments in which the composition is employed as a first shell portion, the second shell portion may comprise any suitable materials, and be applied by any suitable method, for example, those disclosed in published U.S. application 2004-0062804; published US application 2004-0081695 A1; published US application 2004-0146559; and published US application 2003-0219484, the disclosures of which are incorporated herein by reference.

The core may be any solid form. The core can be prepared by any suitable method, including for example compression or molding. As used herein, “core” refers to a material that is at least partially enveloped or surrounded by another material. Preferably, the core is a self-contained unitary object, such as a tablet or capsule. Typically, the core comprises a solid, for example, the core may be a compressed or molded tablet, hard or soft capsule, suppository, or a confectionery form such as a lozenge, nougat, caramel, fondant, or fat based composition. In certain other embodiments, the core or a portion thereof may be in the form of a semi-solid in the finished dosage form. For example the core may comprise a semisolid fondant material.

In one embodiment the core is a compressed tablet having a hardness from about 2 to about 30 kp/cm², e.g. from about 6 to about 25 kp/cm². “Hardness” is a term used in the art to describe the diametral breaking strength of either the core or the coated solid dosage form as measured by conventional pharmaceutical hardness testing equipment, such as a Schleuniger Hardness Tester. In order to compare values across different size tablets, the breaking strength must be normalized for the area of the break. This normalized value, expressed in kp/cm², is sometimes referred in the art as tablet tensile strength. A general discussion of tablet hardness testing is found in Leiberman et al., Pharmaceutical Dosage Forms—Tablets, Volume 2, ₂nd Ed., Marcel Dekker Inc., 1990, pp. 213-217, 327-329.

The core may have one of a variety of different shapes. For example, the core may be shaped as a polyhedron, such as a cube, pyramid, prism, or the like; or may have the geometry of a space figure with some non-flat faces, such as a cone, truncated cone, cylinder, sphere, torus, or the like. In certain embodiments, a core has one or more major faces. For example, in embodiments wherein a core is a compressed tablet, the core surface typically has two opposing major faces formed by contact with the upper and lower punch faces in the compression machine. In such embodiments the core surface typically further comprises a “belly-band” located between the two major faces, and formed by contact with the die walls in the compression machine. A core may also comprise a multilayer tablet. Exemplary core shapes that may be employed include tablet shapes formed from compression tooling shapes described by “The Elizabeth Companies Tablet Design Training Manual” (Elizabeth Carbide Die Co., Inc., p. 7 (McKeesport, Pa.) (incorporated herein by reference).

The core typically comprises active ingredient and a variety of excipients, depending on the method by which it is made.

In embodiments in which the core is made by compression, suitable excipients include fillers, binders, disintegrants, lubricants, glidants, and the like, as known in the art. A core made by compression may be a single or multi-layer, for example bi-layer, tablet.

Suitable fillers for use in making the core by compression include water-soluble compressible carbohydrates such as sugars, which include dextrose, sucrose, maltose, and lactose, sugar-alcohols, which include mannitol, sorbitol, maltitol, xylitol, starch hydrolysates, which include dextrins, and maltodextrins, and the like, water insoluble plastically deforming materials such as microcrystalline cellulose or other-cellulosic derivatives, water-insoluble brittle fracture materials such as dicalcium phosphate, tricalcium phosphate and the like and mixtures thereof.

Suitable binders for making the core by compression include dry binders such as polyvinyl pyrrolidone, hydroxypropylmethylcellulose, and the like; wet binders such as water-soluble polymers, including hydrocolloids such as acacia, alginates, agar, guar gum, locust bean, carrageenan, carboxymethylcellulose, tara, gum arabic, tragacanth, pectin, xanthan, gellan, gelatin, maltodextrin, galactomannan, pusstulan, laminarin, scleroglucan, inulin, whelan, rhamsan, zooglan, methylan, chitin, cyclodextrin, chitosan, polyvinyl pyrrolidone, cellulosics, sucrose, starches, and the like; and derivatives and mixtures thereof.

Suitable disintegrants for making the core by compression, include sodium starch glycolate, cross-linked polyvinylpyrrolidone, cross-linked carboxymethylcellulose, starches, microcrystalline cellulose, and the like.

Suitable lubricants for making the core by compression include long chain fatty acids and their salts, such as magnesium stearate and stearic acid, talc, glycerides and waxes.

Suitable glidants for making the core by compression include colloidal silicon dioxide, and the like.

In certain embodiments, the core or a portion thereof may optionally comprise release modifying excipients as known in the art, for example as disclosed in published U.S. application 2004-0062804, the disclosure of which is incorporated by reference herein. Suitable release-modifying excipients for making the core by compression include swellable erodible hydrophilic materials, insoluble edible materials, pH-dependent polymers, and the like.

Suitable pharmaceutically acceptable adjuvants for making the cores by compression include, preservatives; high intensity sweeteners such as aspartame, acesulfame potassium, sucralose, and saccharin; flavorants; colorants; antioxidants; surfactants; wetting agents; and the like and mixtures thereof.

In embodiments wherein the core is prepared by compression, a dry blending (i.e. direct compression), or wet granulation process may be employed, as known in the art. In a dry blending (direct compression) method, the active ingredient or ingredients, together with the excipients, are blended in a suitable blender, then transferred directly to a compression machine for pressing into tablets. In a wet granulation method, the active ingredient or ingredients, appropriate excipients, and a solution or dispersion of a wet binder (e.g. an aqueous cooked starch paste, or solution of polyvinyl pyrrolidone) are mixed and granulated. Alternatively a dry binder may be included among the excipients, and the mixture may be granulated with water or other suitable solvent. Suitable apparatuses for wet granulation are known in the art, including low shear, e.g. planetary mixers; high shear mixers; and fluid beds, including rotary fluid beds. The resulting granulated material is dried, and optionally dry-blended with further ingredients, e.g. adjuvants and/or excipients such as for example lubricants, colorants, and the like. The final dry blend is then suitable for compression. Methods for direct compression and wet granulation processes are known in the art, and are described in detail in, for example, Lachman, et al., The Theory and Practice of Industrial Pharmacy, Chapter 11 (3rd Ed. 1986).

The dry-blended, or wet granulated,.powder mixture is typically compacted into tablets using a rotary compression machine as known in the art, such as for example those commercially available from Fette America Inc., Rockaway, N.J., or Manesty Machines LTD, Liverpool, UK. In a rotary compression machine, a metered volume of powder is filled into a die cavity, which rotates as part of a “die table” from the filling position to a compaction position where the powder is compacted between an upper and a lower punch to an ejection position where the resulting tablet is pushed from the die cavity by the lower punch and guided to an ejection chute by a stationary “take-off” bar.

In one optional embodiment, the core may be prepared by the compression methods and apparatus described in U.S. Pat. No. 6,767,200, the disclosure of which is incorporated herein by reference. Specifically, the core is made using a rotary compression module comprising a fill zone, insertion zone, compression zone, ejection zone, and purge zone in a single apparatus having a double row die construction as shown therein. The dies of the compression module are preferably filled using the assistance of a vacuum, with filters located in or near each die.

The shell may be substantially unitary and continuous, or the shell may comprise multiple portions, e.g. a first shell portion and a second shell portion. In certain embodiments, at least one such shell portion comprises the composition of the invention. In certain embodiments the shell or shell portions are in direct contact with the core. In certain other embodiments, the shell or shell portions are in direct contact with a subcoating that substantially surrounds the core. In certain embodiments, the shell or a shell portion may comprise one ore more openings therein.

In embodiments in which the shell or shell portion is applied to the core by molding, at least a portion of the shell surrounds the core such that the shell inner surface resides substantially conformably upon the core outer surface. As used herein, the term “substantially conformably” shall mean that the inner surface of the shell has peaks and valleys or indentations and protrusions corresponding substantially inversely to the peaks and valleys of the outer surface of the core. In certain such embodiments, the indentations and protrusions typically have a length, width, height or depth in one dimension of greater than 10 microns, say greater than 20 microns, and less than about 30,000 microns, preferably less than about 2000 microns.

In certain embodiments, the shell comprises a first shell portion and a second shell portion that are compositionally different. In one embodiment, a first shell portion comprises the composition of the invention, and a second shell portion is compositionally different from the first shell portion. As used herein, the term “compositionally different” means having features that are readily distinguishable by qualitative or quantitative chemical analysis, physical testing, or visual observation. For example, the first and second shell portions may contain different ingredients, or different levels of the same ingredients, or the first and second shell portions may have different physical or chemical properties, different functional properties, or be visually distinct. Examples of physical or chemical properties that may be different include hydrophilicity, hydrophobicity, hygroscopicity, elasticity, plasticity, tensile strength, crystallinity, and density. Examples of functional properties which may be different include rate and/or extent of dissolution of the material itself or of an active ingredient therefrom, rate of disintegration of the material, permeability to active ingredients, permeability to water or aqueous media, and the like. Examples of visual distinctions include size, shape, topography, or other geometric features, color, hue, opacity, and gloss.

In one embodiment, an aqueous dispersion of the composition comprising the low molecular weight, water soluble polymer and at least one gum capable of forming or contributing to the formation of a thermoreversible gel is used to prepare the shell. In particular, these ingredients are dispersed in water at a temperature above the cloud point of the low molecular weight, water soluble polymer. The dispersion is applied to a core, by for example molding, dipping, spraying, or other means. Preferably, the dispersion is applied to the core by injection molding. Spraying is least preferred. After application of the dispersion to the core, the core is cooled, preferably at a relatively high temperature, i.e., above the cloud point of the low molecular weight, water soluble polymer.

The aqueous dispersion typically comprises about 5 to about 40 weight percent solids. In one embodiment, the aqueous dispersion comprises about 10 to about 30 weight percent solids. In one embodiment, the low molecular weight, water soluble polymer comprises about 8 to about 20 weight percent of the total weight of the aqueous dispersion.

The shell thickness at various locations may be measured using a microscope, for example, an environmental scanning electron microscope, model XL 30 ESEM LaB6, Philips Electronic Instruments Company, Mahwah, Wis. The shell thickness is measured at 6 different locations on a single dosage form. The relative standard deviation (RSD) is calculated as the sample standard deviation, divided by the mean, times 100 as known in the art (i.e. the RSD is the standard deviation expressed as a percentage of the mean). The RSD in shell thickness provides an indication of the variation in the thickness of the shell on a single dosage form. In certain optional embodiments of the invention, the relative standard deviation in shell thickness is less than about 40%, e.g. less than about 30%, or less than about 20%.

The shell itself or an outer coating thereon may optionally contain active ingredient. In one embodiment, such active ingredient will be released immediately from the dosage form upon ingestion, or contacting of the dosage form with a liquid medium.

In certain embodiments of the invention, the core, the shell, or the composition is prepared by molding. In such embodiments, the core, the shell, or the composition is made from a dispersion as described above optionally comprising active ingredient. The dispersion comprises the low molecular weight, water soluble polymer dispersed in a liquid carrier comprising the gum at a temperature above the cloud point of the low molecular weight polymer and above the gelling temperature of the gum component.

In one embodiment, molding is performed via thermal setting molding using the method and apparatus described in U.S. Pat. No. 6,767,200, the disclosure of which is incorporated herein by reference. In this embodiment, the shell is formed by injecting the dispersion into a molding chamber. The dispersion is cooled and solidifies in the molding chamber into a shaped form (i.e., having the shape of the mold).

According to this method, the dispersion may comprise solid particles of the low molecular weight, water-soluble cellulosic polymer suspended in a liquid carrier comprising the other ingredients (the gum component) and optionally a liquid plasticizer.

In one optional embodiment of the invention, the shell is applied to the dosage form using a thermal cycle molding apparatus of the general type shown in published U.S. patent application 2003-0086973 comprising rotatable center mold assemblies, lower mold assemblies and upper mold assemblies. Cores are continuously fed to the mold assemblies. Dispersion for making the shell, which is heated to a flowable state in a reservoir, is injected into the mold cavities created by the closed mold assemblies holding the cores. The temperature of the shell dispersion is then decreased, hardening it around the cores. The mold assemblies open and eject the finished dosage forms. Shell coating is performed in two steps, each half of the dosage forms being coated separately via rotation of the center mold assembly.

In one embodiment, the compression module of U.S. Patent No. 6,767,200 may be employed to make the core. The shell is applied to the core using a thermal cycle molding module as described above. A transfer device as described in published U.S. patent application No. 2003-0066068, the disclosure of which is incorporated herein by reference, may be used to transfer the cores from the compression module to the thermal cycle molding module. The transfer device rotates and operates in sync with the compression module and the thermal cycle molding module to which it is coupled.

In certain optional embodiments the shell, core, or the composition of the invention may additionally comprise a water insoluble polymer at a level of up to about 40%, e.g. 15% of the weight of the shell, core, or the composition of the invention. In embodiments wherein a water insoluble polymer is employed, the weight ratio of low molecular weight water soluble polymer to water insoluble polymer may be from about 99:1 to about 50:50. Suitable water insoluble polymers include ethyl cellulose, cellulose acetate, cellulose acetate butyrate, cellulose propionate, and mixtures thereof.

The dispersion for making cores or the shell by molding may optionally comprise adjuvants or excipients, which may comprise up to about 30% by weight of the dispersion. Examples of suitable adjuvants or excipients include detackifiers, humectants, surfactants, anti-foaming agents, colorants, flavorants, sweeteners, opacifiers, and the like.

In embodiments in which the composition is prepared by molding, the composition typically is preferably substantially free of pores in the diameter range of 0.5 to 5.0 microns, i.e. has a pore volume in the pore diameter range of 0.5 to 5.0 microns of less than about 0.02 cc/g, preferably less than about 0.01 cc/g, more preferably less than about 0.005 cc/g. Typical compressed materials have pore volumes in this diameter range of more than about 0.02 cc/g. Pore volume, pore diameter and density may be determined using a Quantachrome Instruments PoreMaster 60 mercury intrusion porosimeter and associated computer software program known as “Porowin.” The procedure is documented in the Quantachrome Instruments PoreMaster Operation Manual. The PoreMaster determines both pore volume and pore diameter of a solid or powder by forced intrusion of a non-wetting liquid (mercury), which involves evacuation of the sample in a sample cell (penetrometer), filling the cell with mercury to surround the sample with mercury, applying pressure to the sample cell by: (i) compressed air (up to 50 psi maximum); and (ii) a hydraulic (oil) pressure generator (up to 60000 psi maximum). Intruded volume is measured by a change in the capacitance as mercury moves from outside the sample into its pores under applied pressure. The corresponding pore size diameter (d) at which the intrusion takes place is calculated directly from the so-called “Washburn Equation”.

The following non-limiting examples further illustrate the invention.

### EXAMPLES

Dosage forms of this invention are prepared by the method as described below:

A. Preparation of Dispersion for Making a Shell:

### Example 1

Dispersion is prepared containing 50 parts of hydroxypropyl methylcellulose (HPMC) having a viscosity of about 3 cps in 2% aqueous solution [commercially available from Dow Chemical as METHOCEL K3]; 45 parts of Kappa Carrageenan, and 5 parts of glycerin in 900 parts of purified water. The solution has non-volatiles concentration about 10%. First, the purified water is pre-heated to 65° C. with agitation by an electrical mixer equipped with a propeller style blade. At 65° C., the HPMC powder and carrageenan powder are added orderly to the water to form an aqueous dispersion. With continued mixing, the mixture is heated to 80-85° C. to dissolve the carrageenan while the HPMC remains as a dispersed solid. Finally, the glycerin is added to the carrageenan-based dispersion as a plasticizer.

### Example 2

Dispersion is prepared containing 39 parts of hydroxypropyl methylcellulose (HPMC) having a viscosity of about 3 cps in 2% aqueous solution [commercially available from Dow Chemical as METHOCEL K3]; 35 parts of Kappa Carrageenan, 1 part of stearol macrogol-32 diglycerides (Gelucire 50/13) as a anti-foaming agent, 10 parts of sodium carboxymethylcellulose (low molecular weight) as a dispersant, 5 parts of locust bean gum as a wet-gel enhancer, and 10 parts of glycerin in 900 parts of purified water. The solution has non-volatiles concentration about 10%. First, the Gelucire 50/13, sodium carboxymethylcellulose and locust bean gum are added to the purified water. The mixture is then heated to 65° C. with agitation by an electrical mixer equipped with a propeller style blade. At 65° C., the HPMC powder and the carrageenan powder are added orderly to the water system to form an aqueous dispersion. With continued mixing, the mixture is heated to 80-85° C. to dissolve the carrageenan while the HPMC remains as a dispersed solid. Finally, the glycerin is added to the carrageenan-based dispersion as a plasticizer.

### Example 3

Dispersion is prepared containing 10 parts of hydroxypropyl methylcellulose (HPMC) having a viscosity of about 3 cps in 2% aqueous solution [commercially available from Dow Chemical as METHOCEL K3]; 28 parts of Kappa Carrageenan, 1 part of Gelucire 50/13 as a anti-foaming agent, 2 parts of sodium carboxymethylcellulose (low molecular weight) as a dispersant, 7 parts of locust bean gum as a wet-gel enhancer, 9 parts of low-substituted Hydroxypropyl cellulose (L-HPC) and 3 parts of glycerin in 940 parts of purified water. The solution has non-volatiles concentration about 6%. First, Gelucire 50/13, sodium carboxymethylcellulose and locust bean gum is added to the purified water. The mixture is then heated to 65° C. with agitation by an electrical mixer equipped with a propeller style blade. At 65° C., the powders of HPMC, L-HPC and carrageenan are added orderly to the water system to form an aqueous dispersion. With continued mixing, the mixture is heated to 80-85° C. to dissolve the carrageenan while the HPMC remains as a dispersed solid. Finally, the glycerin is added to the carrageenan-based dispersion as a plasticizer.

When gelled films of samples from Examples 1-3 are placed on to a flat surface, no discharge of water is observed, indicating that a minimal amount to no water is released into a moisture sensitive tablet.

Example 5 (Control without addition of low molecular weight cellulosic polymer) The coating formulation is prepared containing 32 parts of Kappa Carrageenan and 8 parts of Locust bean gum in 960 parts of purified water. The solution has non-volatiles concentration about 4%. First, locust bean gum powder and carrageenan powder are added orderly to the water with agitation by an electrical mixer equipped with a propeller style blade to form an aqueous dispersion. With continued mixing, the mixture is heated to 80-85° C. to dissolve the carrageenan and locust bean gum.

When gelled film samples of Example 4 are placed on to a flat surface, a discharge of water is observed, indicating the potential release of water into a moisture sensitive tablet.

B. Applying the Shell to Cores:

The hot dispersions (˜70-80° C.) from Part A are applied to cores (i.e. Acetaminophen 500 mg tablet cores as in Table 1) by a single injection to obtain dosage forms having shells residing upon the cores. The cores are compressed to a hardness of 9-14 kiloponds using a rotary tablet press. First, the cores are transferred into a molding chamber. Next, the hot dispersion from Part A is injected into the molding chamber to surround the tablet and to form shell by cooling. The mold temperature is set around 30-35° C. The shell has excellent strength, and is easily removed from the mold. The coated tablets are then dried by a mechanical drier at 23-25° C. and 30-35% RH.

TABLE 1
Acetaminophen Core Formulation

| | Percent | |
|---|---|---|
| Ingredients | (w/w) | mg/tab |
| Acetaminophen USP | 82.89 | 500.0 |
| Powdered Cellulose NF | 6.63 | 40.0 |
| Sodium Starch Glycolate NF | 1.66 | 10.0 |
| Pregelatinized Starch NF | 1.66 | 10.0 |
| Starch (Cornstarch)NF | 6.63 | 40.0 |
| Magnesium Stearate NF | 0.53 | 3.20 |
| TOTAL | 100.0 | 603.2 |

C. Dissolution Study

The dissolutions for acetaminophen were analyzed using the following dissolution analysis: USP Type II apparatus (paddles, 50 RPM) in monobasic sodium phosphate at 37° C. Approximately 10 mL samples were pulled for analysis at the 30 minute timepoint. Dissolution samples were analyzed for acetaminophen versus a standard prepared at the theoretical concentration for 100% released. Samples were analyzed using an Agilent® UV spectrophotometer set at a wavelength of 243 nm for the acid stage using a 0.02 cm flow-cell.

What is claimed:

1. A composition comprising:
a) a shell-forming component comprising
i) a low-molecular weight water-soluble polymer
ii) at least one gum capable of forming or contributing to the
formation of thermoreversible gel; and
wherein the water-soluble polymer has a cloud point in an aqueous system within a temperature range of about 20° C. and about 90° C.

2. A composition according to claim 1 wherein the at least one gum is a blend of gums capable of forming or contributing to the formation of thermoreversible gel that is at least 50% by weight of a Kappa-carrageenan.

3. A composition according to claim 1 wherein the shell-forming component comprises 20 to 75 weight percent of the low molecular weight, water-soluble polymer as a percentage of the dried film and 25 to 80 weight percent of the at least one gum as a percentage of the dried film.

4. A composition according to claim 1, wherein the low molecular weight, water soluble polymer is selected from the group consisting of hydroxypropylmethyl cellulose, hydroxypropyl cellulose, methyl cellulose and mixtures thereof.

5. A composition according to claim 1, wherein the low molecular weight, water soluble polymer comprises hydroxypropyl methylcellulose having a viscosity from about 3 to about 80 mPa s in 2% aqueous solution at 25° C.

6. A composition according to claim 1, wherein the low molecular weight, water soluble polymer comprises hydroxypropyl methylcellulose having a viscosity from about 3 to about 50 mPa s in 2% aqueous solution at 25° C.

7. A composition according to claim 1, wherein the low molecular weigh,:, water soluble polymer comprises at least 75% by weight of the total weight of water soluble polymer in the composition as hydroxypropyl methylcellulose having a viscosity from about 3 to about. 50 mPa s in a 2% aqueous solution at 25° C.

8. The composition according to claim 1 wherein the shell-forming component further comprises a gelling salt.

9. The composition according to claim 1 wherein a percentage of active ingredient dissolved from the finished dosage form after application and drying of the shell is not less than 90% of the active ingredient dissolved at any time point of an equivalent uncoated core, according to a preferred method for said active.

10. The composition according to claim 1 wherein a percentage of active ingredient dissolved from the finished dosage form upon storage conditions of 40° C. and 75% relative humidity for up to 6 months is not less than 90% of active ingredient dissolved at any time point of an equivalent uncoated core, according to a preferred method for said active.

11. The composition according to claim 1 wherein the degradation of the active ingredient is not more than 1% as measured by the chemically degraded derivative compound of the active ingredient upon application and drying of the shell.

12. The composition according to claim 1 wherein the degradation of the active ingredient is not more than 1% as measured by the chemically degraded derivative compounds of the active ingredient at storage conditions of 40° C. and 75% relative humidity for up to 6 months.

13. The composition according to claim 1, wherein the water-soluble polymer has a cloud point in an aqueous system within a temperature range of about 30° C. and about 80° C.

14. The composition according to claim 1, wherein the water-soluble polymer has a cloud point in an aqueous system within a temperature range of about 35° C. and about 70° C.

15. A dosage form comprising a shell that comprises
a) a low-molecular weight water-soluble polymer and
b) at least one gum capable of forming or contributing to the formation of thermoreversible gel,
wherein the water-soluble polymer has a cloud point in an aqueous system within a temperature range for the aqueous system of about 20° C. and about 90° C.

16. The dosage form according to claim 15, wherein the water-soluble polymer has a cloud point in an aqueous system within a temperature range of about 30° C. and about 80° C.

17. The dosage form according to claim 15, wherein the water-soluble polymer has a cloud point in an aqueous system within a temperature range of about 35° C. and about 70° C.

18. A dosage form according to claim 15 further comprising a core, wherein the core comprises at least one active ingredient.

19. A dosage form according to claim 15 wherein the at least one gum is a blend of gums capable of forming or contributing to the formation of thermoreversible gel that is at least 50% by weight of a Kappa-carrageenan.

20. A dosage form of claim 15 wherein the core comprises a compressed tablet.

21. A dosage form according to claim 15 wherein the shell-forming component comprises 20 to 75 weight percent of the low molecular weight, water-soluble polymer as a percentage of the dried film and 25 to 80 weight percent of the at least one gum as a percentage of the dried film.

22. A dosage form according to claim 15, wherein the low molecular weight, water soluble polymer is selected from the group consisting of hydroxypropylmethyl cellulose, hydroxypropyl cellulose, methyl cellulose and mixtures thereof.

23. A dosage form according to claim 15, wherein the low molecular weight, water soluble polymer comprises hydroxypropyl methylcellulose having a viscosity from about 3 to about 80 mPa s in 2% aqueous solution.

24. A dosage form according to claim 15, wherein the low molecular weight, water soluble polymer comprises hydroxypropyl methylcellulose having a viscosity from about 3 to about 50 mPa s in 2% aqueous solution.

25. A dosage form according to claim 15, wherein the low molecular weight, water soluble polymer comprises at least 75% by weight of the total weight of water soluble polymer in the composition as hydroxypropyl methylcellulose having a viscosity from about 3 to about 50 mPa s in 2% aqueous solution.

26. A process for preparing a dosage form comprising coating a core containing a pharmaceutical active ingredient with the composition according to claim 1.

27. A process for preparing a core and shell dosage form comprising
a) forming a compressed core containing at least one pharmaceutical active ingredient in compression tableting machine; and
b) coating the compressed core with the composition according to claim 1.

28. A process for preparing a core and shell dosage form comprising
a) forming a solid, compressed core containing at least one pharmaceutical active ingredient in a tableting machine;
b) introducing the compressed core into a mold cavity; and
c) injecting the composition according to claim 1 into the mold cavity to coat at least a portion of the compressed core.

29. A process for preparing a core and shell dosage form comprising
a) forming a solid, compressed core containing at least one pharmaceutical active ingredient in a tableting machine;
b) introducing the compressed core into a mold cavity;
c) injecting the composition according to claim 1 into the mold cavity to coat at least a portion of the compressed core;
d) rotating the mold cavity; and
e) injecting a liquid curable composition into said mold to coat at least a second portion of the compressed core.

30. A dosage form comprising a core, having a shell at least on a portion thereof, wherein the shell has a thickness from about 10 to about 80 microns, and wherein said dosage form is prepared by a process comprising
a) introducing said core into a mold cavity
b) injecting the composition of claim 1 into the mold cavity to coat at, least a portion of the core

31. A dosage form prepared by process in claim 30 wherein the dosage form is ejected from the mold cavity following step b, and the injection of said composition and ejection of the dosage form takes 6 seconds or less.

32. A composition consisting essentially of: a) 20 to 75 weight percent of hydroxypropyl methylcellulose having a viscosity from about 3 to about 80 mPa s in 2% aqueous solution; b) 25 to 80 weight percent of a gum component comprising at least 50% by weight of Kappa-carrageenan.

33. An aqueous dispersion comprising: a) 1 to 11 weight percent of a low molecular weight, water soluble polymer that has a cloud point in an aqueous systems within a temperature range for the aqueous system of about 20° C. and about 80° C.; b) 1.3 to 12 weight percent of a gum component comprising at least 50% by weight of Kappa-carrageenan; and d) about 85-95 weight percent water.
